(19) United States
(12) Patent Application Publication
(10) Pub. No.: US 2021/0085413 A1
(43) Pub. Date: Mar. 25, 2021

(54) PATIENT FACE SHEET

(71) Applicant: Purifas Holdings Pty Ltd, Bankstow (AU)

(72) Inventor: Ibrahim Samaan, Bankstown (AU)

(21) Appl. No.: 16/499,100

(22) PCT Filed: Jan. 31, 2018
(86) PCT No.: PCT/AU2018/050060

§ 371 Date: Sep. 27, 2019

(30) Foreign Application Priority Data
Mar. 30, 2017 (AU) 2017202118

(51) Int. Cl. A61B 46/00; A61G 13/12; A61G 13/00; A61G 15/00; A61G 15/12

(52) U.S. Cl. CPC A61B 46/00; A61G 13/121; A61G 2200/325; A61G 15/007; A61G 15/125; A61G 13/009

(57) ABSTRACT

The present disclosure relates generally to a patient face sheet for a therapy treatment table including a headrest. The patient face sheet may be used in conjunction with an optional pillow including a face opening which is positioned to align with the corresponding opening in the headrest. The patient face sheet comprises a main panel to which a protective skirt is connected. The main panel includes a central and generally circular-shaped opening about which the protective skirt is connected. The protective skirt is draped within both the face hole and the corresponding hole to provide substantially full shielding of the optional pillow and the headrest from the patient's head.

### CROSS-REFERENCE TO RELATED APPLICATIONS

The instant application claims priority to Australian Patent Application No. 2017202118 filed Mar. 30, 2017 and PCT Application No. PCT/AU2018/050060, filed Jan. 31, 2018, both applications are titled Patient Face Sheet, the entire contents of which are incorporated herein by reference.

### TECHNICAL FIELD

The present disclosure relates broadly to a patient face sheet for a treatment table or chair, such as a massage or physiotherapy table or chair.

### BACKGROUND OF DISCLOSURE

It is common practice to use patient face sheets with massage or other therapy treatment tables or chairs. The patient face sheet is designed to promote hygienic practice in protecting both the practitioner and the patient from bacteria, microbes, sweat, saliva, makeup or other contaminants associated with the shared surfaces of the treatment table or chair. The conventional face sheet is typically constructed flat in one piece from non-woven material with a face opening or a cross-slit in its middle region. The cross-slit allows a rough face opening to be formed in the face sheet to provide largely unobstructed breathing for the patient when lying face down via the face hole of the treatment table or chair. These types of face sheets suffer from at least the following drawbacks:

i) it is difficult to form the opening via the cross-slit in such a way that it both avoids direct facial contact with the table or chair and provides a clear passage through the face hole;

ii) they are relatively ineffective in protecting or shielding the patient from contaminants.

### SUMMARY OF DISCLOSURE

According to the present disclosure there is provided a patient face sheet for a therapy treatment table or chair, said face sheet comprising:

a main panel including a central opening being adapted to locate at a face hole associated with the treatment table or chair;

a protective skirt connected to the main panel about the central opening, said skirt adapted to locate within the face hole of the treatment table or chair.

The protective skirt may be continuous about its perimeter and configured to drape within the face hole. Additionally or alternatively, the continuous skirt may be of sufficient length to provide substantially full shielding of the peripheral surface of the face hole from the patient's head.

The protective skirt may be connected with the main panel. Additionally or alternatively, the protective skirt may be connected continuous with the main panel.

The face sheet may be constructed of a non-woven fabric and is disposable. Alternatively, the patient face sheet may be constructed of a launderable material and is reusable.

### BRIEF DESCRIPTION OF DRAWINGS

In order to achieve a better understanding of the nature of the present disclosure a preferred embodiment of a patient face sheet for a therapy treatment table or chair will now be described, by way of example only, with reference to the accompanying drawings in which:

FIG. 1 is a perspective and partly exploded view of a patient face sheet of one embodiment of the disclosure installed on a typical therapy treatment table;

FIG. 2 is a perspective view from above of the patient face sheet of FIG. 1 shown together with a patient in a prone position;

FIG. 3 is an exploded perspective view from below of the patient face sheet of the preceding figures together with an optional pillow used in conjunction with the therapy treatment table;

FIG. 4 is a perspective view from below of the patient face sheet and the optional pillow of the preceding figures.

### DETAILED DESCRIPTION

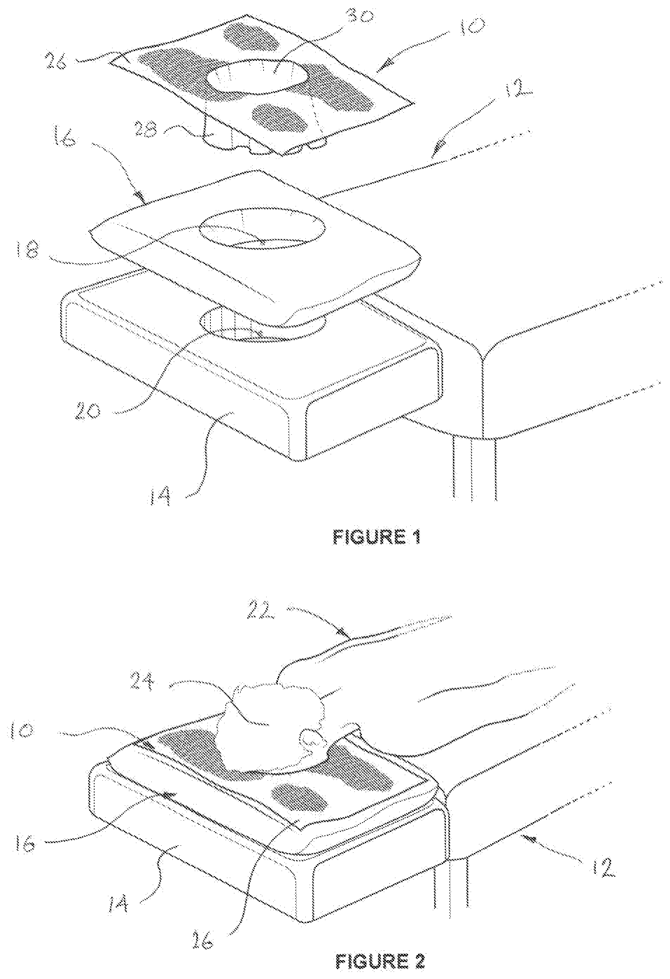

As shown in FIGS. 1 and 2 there is a patient face sheet 10 of one embodiment of the disclosure for a therapy treatment table 12 including a headrest 14. The patient face sheet 10 may be used in conjunction with a pillow 16 including a face opening 18 which is positioned to align with a corresponding opening 20 in the headrest 14. The face sheet 10 may also be designed for use with a therapy treatment chair.

As shown in FIG. 2 a patient such as 22 lies in a prone position with their head 24 generally aligned with the face hole 18 of the optional pillow 16 and the corresponding hole 20 in the headrest 14. The patient face sheet 10 is disposed between the patient's head 24 and the optional pillow 16. The patient face sheet 10 is designed to prevent direct contact of the patient's head 24 with the pillow 16 or, in the absence of the pillow 16, with the headrest 14. Rather, the patient's head 24 is in direct contact with the face sheet 10 alone. The face sheet 10 thus provides shielding of the pillow 16 and/or the headrest 14 from contaminants associated with the patient 22 such as but not limited to bacteria, microbes, sweat, saliva and makeup.

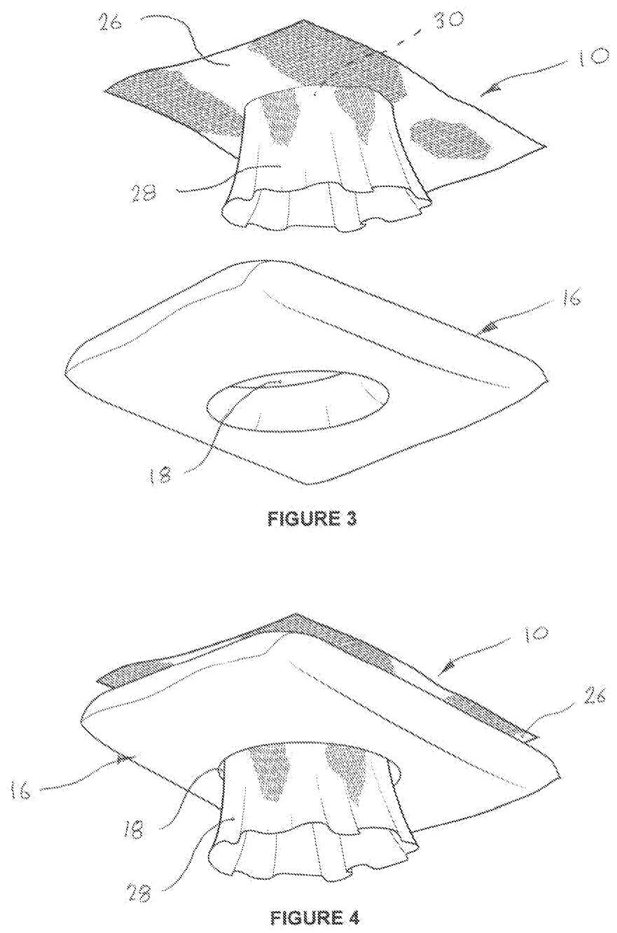

In this embodiment and as best seen in FIGS. 3 and 4 the patient face sheet 10 comprises a main panel 26 to which a protective skirt 28 is connected. The main panel 26 is shaped generally rectangular and provides coverage across substantially the full upper surface of the optional pillow 16. The main panel 26 includes a central and generally circular-shaped opening 30 about which the protective skirt 28 is connected.

In this embodiment the protective skirt 28 is draped within both the face hole 18 of the optional pillow 16 and the corresponding hole 20 in the headrest 14. The protective skirt 28 is of sufficient length or depth to provide substantially full shielding of the optional pillow 16 and the headrest 14 from the patient's head 24 and associated contaminants. The skirt 28 is configured to sacrificially capture the contaminants and can be either disposed of or laundered for subsequent use.

In this embodiment the protective skirt 28 is both continuous about its perimeter and is connected continuous with the main panel 26. The patient face sheet 10 may be constructed of either a non-woven fabric which is disposable, or a launderable material which is reusable. Unlike the prior art, the face sheet 10 is three-dimensional having the skirt 28 draped in a third dimension from the otherwise two-dimensional main panel 26. This 3D design introduces difficulty in terms of both its manufacture and packaging. In order to manufacture the face sheet 10 with the third dimension of the protective skirt 28, preferably being continuous about its perimeter, the skirt 28 will most likely be formed as a separate component to the main panel 26 and thereafter connected continuous with the main panel 26. In packaging the face sheet 10 it is preferable that the skirt 28 collapses or folds toward or generally into the same plane as the main panel 26 for compact packaging. For this purpose, the skirt 28 as best seen in FIGS. 3 and 4 tapers outward in a direction away from the opening 30 and thus is capable of collapsing outward and substantially into the plane of the main panel 26 for flat packing.

It is to be understood that the patient face sheet can be used in all forms of treatment, and in particular, therapy treatment including but not limited to physiotherapy, chiropractic, osteotherapy, massage, acupuncture, naturopath and laser treatment. It is also to be understood that patient in the context of the patient face sheet is to extend to any person including a client or customer.

Now that a preferred embodiment of a patient face sheet has been described, it will be apparent to those skilled in the art that it has the following advantages:

1. it provides substantially full coverage about the otherwise exposed surfaces of the therapy treatment table or chair and in particular the optional head pillow and/or headrest about their respective openings to reduce the likelihood of transmitting bacteria, microbes and other contaminants to patients;

2. the patient face sheet provides relatively clear vision for the patient which is particularly important in the event the patient has claustrophobic tendencies;

3. the patient face sheet can be quickly and effectively located in a therapy treatment table or chair with or without an optional head pillow.

Those skilled in the art will appreciate that the disclosure as described herein is susceptible to variations and modifications other than those specifically described. For example, the protective skirt may be discontinuous provided it substantially shields the patient's head from likely contact with contamination surfaces of the therapy treatment table or chair. The protective skirt may also vary in its length or third dimensional depth. The main panel may be of practically any shape provided it also shields the patient's head from contact surfaces of the therapy table or the associated optional pillow. The opening in the main panel of the face sheet may extend to other shapes including but not limited to oval or elliptical shaped openings. All such variations and modifications are to be considered within the scope of the present disclosure the nature of which is to be determined from the foregoing description.

1. A patient face sheet for a therapy treatment table or chair, said face sheet comprising:
a main panel including a central opening being adapted to locate at a face hole associated with the treatment table or chair;
a protective skirt connected to the main panel about the central opening, said skirt adapted to locate within the face hole of the treatment table or chair.

2. The patient face sheet of claim 1 wherein the protective skirt is continuous about its perimeter and configured to drape within the face hole.

3. The patient face sheet of claim 2 wherein the continuous skirt is of sufficient length to provide substantially full shielding of the peripheral surface of the face hole from the patient's head.

4. The patient face sheet of claim 1 wherein the protective skirt is connected with the main panel.

5. The patient face sheet of claim 2 wherein the protective skirt is connected with the main panel.

6. The patient face sheet of claim 4 wherein the protective skirt is connected continuous with the main panel.

7. The patient face sheet of claim 1 wherein the patient face sheet is constructed of a non-woven fabric and is disposable.

8. The patient face sheet of claim 1 wherein the face sheet is constructed of a launderable material and is reusable.
